(19) United States
(12) Patent Application Publication
(10) Pub. No.: US 2020/0230579 A1
(43) Pub. Date: Jul. 23, 2020

(54) A PLATINUM-GALLIUM BASED ALKANE DEHYDROGENATION CATALYST CONTAINING AN OXIDATION PROMOTER

(71) Applicant: HALDOR TOPSØE A/S, Kgs. Lyngby (DK)

(72) Inventors: Rasmus Munksgård NIELSEN, Måløv (DK); Roy VAN DEN BERG, Copenhagen O (DK); Lived J. LEMUS-YEGRES, Copenhagen S (DK); Joachim Harteg JACOBSEN, Birkerød (DK); Poul Erik HØJLUND NIELSEN, Fredensborg (DK)

(73) Assignee: HALDOR TOPSØE A/S, Kgs. Lyngby (DK)

(21) Appl. No.: 16/630,166

(22) PCT Filed: Jun. 28, 2018
(86) PCT No.: PCT/EP2018/067390

§ 371 Date: Jan. 10, 2020

(30) Foreign Application Priority Data
Jul. 13, 2017 (DK) PA 2017 00413

(51) Int. Cl. B01J 23/62; B01J 23/10; B01J 21/12; C07C 5/333; B01J 37/02; B01J 37/08

(52) U.S. Cl. CPC B01J 23/62; B01J 23/10; B01J 21/12; C07C 5/3337; B01J 37/0201; B01J 37/0236; C07C 2523/63; C07C 2521/04; C07C 2523/04; C07C 2523/08; C07C 2523/10; C07C 2523/42; B01J 37/088

(57) ABSTRACT

A platinum-gallium based catalyst for alkane dehydrogenation is provided with an oxidation promotor in the form of cerium that is added to the catalyst composition to improve the regeneration thereof. The cerium is preferably added to the catalyst composition in an amount from 0.001 to 0.5 wt %.

The present invention relates to an oxidation promotor for platinum-gallium based catalysts for alkane dehydrogenation, especially propane dehydrogenation (PDH). More specifically, the invention concerns a platinum-gallium based alkane dehydrogenation catalyst containing an oxidation promotor in the form of cerium that is added to the catalyst composition to improve the regeneration thereof.

Today there are four major processes for alkane dehydrogenation in commercial use. The differences between these processes are primarily concerned with supply of the heat of reaction. The important Catofin process is characterized by the heat of reaction being supplied by pre-heating of the catalyst. The Catofin process is carried out in 3 to 8 fixed bed adiabatic reactors, using a chromium oxide/alumina catalyst containing around 20 wt % chromium oxide. The catalyst may be supplemented with an inert material having a high heat capacity, or alternatively with a material which will selectively combust or react with the hydrogen formed, the so-called heat generating material (HGM). Promoters such as potassium may be added.

The Catofin process is a well-established process and still the dominant industrial process for alkane dehydrogenation. Since the reaction heat is supplied by the catalyst, a sequential operation is used, during which the catalyst bed is used for dehydrogenation. Then the gas is purged away, and the catalyst is being regenerated/heated and the Cr(VI) oxide reduced with hydrogen. Finally, the bed is purged with steam before the next dehydrogenation.

Conventional catalyst regeneration processes often do not sufficiently restore the catalytic activity of platinum-gallium based alkane dehydrogenation catalysts to a level equalling that of such catalysts when they are fresh. Thus, skilled persons who practise alkane dehydrogenation, especially PDH, know that decreasing activity of the catalyst inevitably leads to decreasing alkene production, eventually to a point where process economics dictate replacement of the deactivated catalyst with fresh catalyst. Therefore, means and methods to restore catalyst activity more fully are desirable.

To regenerate platinum-gallium based catalysts for alkane dehydrogenation, an oxidation treatment is required. Typically, high temperatures and long reaction times (up to 2 hours) are needed to fully reactivate the catalysts.

The current commercial catalysts for the Catofin process are based on chromium. Such Cr catalysts require an oxidation treatment to remove built-up coke, but do not require an oxidation treatment to reactivate themselves. The coke removal is generally done by contacting the catalysts with air or another oxygen-containing gas under high temperature conditions.

Prolonged reaction times, high temperatures (up to 650° C.) and high O₂ partial pressures during a regeneration step have proven beneficial for the performance of platinum-gallium based catalysts for propane dehydrogenation in the subsequent propane dehydrogenation cycle. A comparison of these catalysts with current commercial chromium catalysts has shown that the Pt/Ga catalyst outperforms the Cr catalyst in the first cycle, but that Cr has a better steady-state performance during later cycles. The drop for the Pt/Ga catalyst from the first cycle to later cycles is due to an insufficient regeneration/oxidation.

It has now turned out that cerium (Ce) acts as an oxidation promotor for catalyzing the oxidation step, and thereby cerium becomes capable of reactivating platinum-gallium based catalysts faster.

The addition of Ce to the catalyst improves the catalyst reactivation and thereby limits the catalyst deactivation caused by incomplete regeneration. This improved reactivation behavior is very important for commercial applications, because the regeneration time in industrial Catofin plants is typically less than 20 minutes. A more complete regeneration will thus ensure that the catalytic activity remains high, leading to the Catofin plant output remaining high over time.

The use of cerium in connection with catalytic alkane dehydrogenation is described in a number of publications. Thus, US 2004/0029715 deals with the regeneration of a dehydrogenation catalyst containing cerium oxide, and in U.S. Pat. No. 9,415,378, a dehydrogenation catalyst is described, in which the support contains a cerium source.

J. Im & M. Choi, ACS Catal. 6, 2819-2826 (2016) discloses a platinum-gallium based catalyst for propane dehydrogenation to propene, which contains an oxidation promotor in the form of cerium which is added to the catalyst composition in an amount of 0.5-2 wt %. The catalyst is regenerated at a temperature of 620° C. This catalyst is, however, performing better in the Oleflex process, where the Pt needs a treatment with Cl in order to be re-dispersed.

WO 2010/133565 discloses various monolith catalysts that can contain cerium, which e.g. can be used for dehydrogenation. In WO 2004/052535, a calcinated catalyst, especially for dehydrogenating aromatic hydrocarbons, is disclosed. It may contain cerium as a selectivity improver.

The use of rare earth metals as oxidative dehydrogenation catalysts is described in WO 2004/033089, and a catalyst composition and a reactivation process useful for alkane dehydrogenation is disclosed in US 2015/0202601. The catalyst comprises a group IIIA metal such as Ga, a group VIII noble metal such as Pt or Pa, a dopant and an optional promotor metal on a catalyst support which can be e.g. alumina modified by a rare earth metal.

Finally, US 2017/0120222 discloses transition metal/noble metal complex oxide catalysts for dehydrogenation. More specifically, this document describes a procedure of making an improved catalyst performance using a sol-gel method in which a clear positive effect of adding Ce is seen. Results are shown in graphs where the sol-gel using Ce displays a clearly higher conversion than the samples without Ce. For an impregnated sample, the same effect is vaguely seen for C3 dehydrogenation and hardly observable for C4 dehydrogenation. The catalyst has Pt as the active material on a carrier consisting of alumina doped with Ga. The Ce is proposed to stabilize the Pt. So the catalyst described in US 2017/0120222 is also performing better in the Oleflex process, where the Pt needs a treatment with Cl in order to be re-dispersed.

The present invention relates to a platinum-gallium based catalyst for the dehydrogenation of lower alkanes, whereby the alkanes are dehydrogenated to the corresponding alkenes according to the reaction

CₙH₂ₙ₊₂<->CₙH₂ₙ+H₂

in which n is an integer from 2 to 5, by feeding the alkane to a catalyst-containing dehydrogenation reactor, wherein
the catalyst is based on optionally Si-doped alumina that has been impregnated with gallium and platinum, and
Cerium in an amount from 0.001 to 0.5 wt % is added to the catalyst as an oxidation promotor together with gallium and platinum, thereby improving the regeneration of the catalyst composition.

The preferred amount of cerium added to the catalyst is in the range between 0.05 and 0.1 wt %. The cerium can be added as a salt, such as Ce (NO₃)₂.6H₂O.

Preferably, the cerium is added by impregnation together with gallium and platinum. Furthermore, it is preferred that the amount of platinum impregnated into the catalyst composition is up to around 200 ppm.

The effect observed when using a catalyst according to the invention for alkane dehydrogenation is different from that observed according to US 2017/0120222. More specifically, a clear effect on the regeneration efficiency is seen when Ce is added. In fact, by adding just 0.05 wt % Ce, a significantly faster reactivation of the catalyst is observed as compared to a sample without added Ce. Any significant change in the conversion is not seen when the catalyst is fully reactivated. This is highly important for the Catofin process, because the reactivation is done quite frequently and the reactivation time is very short (a few minutes).

The effect is also different from that obtained according to US 2015/0202601. The catalyst used in that document offers a decreased regeneration time under ‘air soak’ in comparison with otherwise identical catalysts. More specifically, the effect is observed for Fe, Cr and V, not for Ce, and a temperature of at least 660° C. is required, whereas according to the present invention, a beneficial effect of Ce is observed at temperatures below 630° C.

It is known that high temperatures (up to around 650° C.) and high O₂ partial pressures during a long regeneration step are beneficial for the performance of a platinum-gallium (Pt/Ga) based catalyst in the next propane dehydrogenation cycle. Experimental testing of such Pt/Ga catalysts versus current commercial Cr catalysts has shown that while the Pt/Ga catalyst outperforms the Cr catalyst in the first cycle, then in later cycles the Cr catalyst shows a better steady-state performance than the Pt/Ga catalyst. The drop of the Pt/Ga catalyst from the first cycle to later cycles is due to an insufficient regeneration/oxidation. Thus, the ability of cerium to catalyze the oxidation step has been investigated and was found to be outstanding.

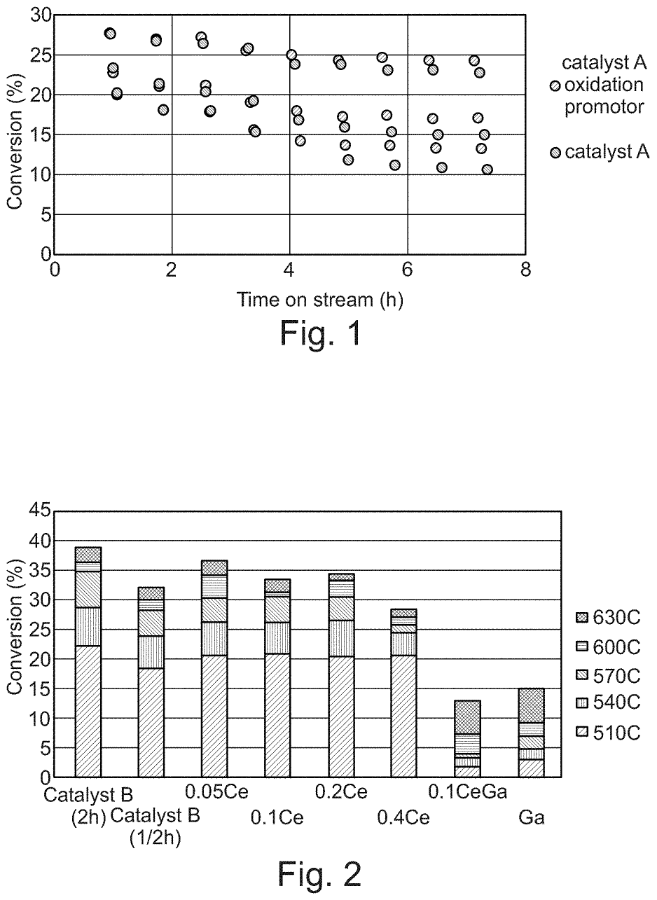

The invention is illustrated further by the examples which follow. In the examples, reference is made to FIGS. 1 and 2, where

FIG. 1 illustrates the impact of cerium on the regeneration procedure, and

FIG. 2 shows the activity of catalysts with and without cerium.

### EXAMPLE 1

This example illustrates the synthesis of a catalyst including the oxidation promotor according to the invention. The synthesis is carried out by co-impregnating approximately 0.1 wt % Ce together with approximately 50 ppm Pt, 1 wt % Ga and 0.2 wt % K on an alumina carrier.

More specifically, a mixture of 4 g of a 5% Ga solution in HNO₃, 0.2 g of a 0.5 wt % Pt solution (Pt(NH₃)₄(HCO₃)₂), 0.062 g of Ce(NO₃)₂.6H₂O and 0.05 g KNO₃ is diluted with 11 g water. The resulting solution is used to impregnate 20 g of gamma/theta Al₂O₃ (spheres, 1000° C., pore volume 0.75 ml/g). The sample is rolled for 1 hour, dried overnight and calcined at 700° C. for 2 hours with a heating ramp of 4 hours.

The effect of Ce on the catalyst regeneration is described in the below examples 2 and 3.

### EXAMPLE 2

The impact of cerium on the regeneration is illustrated in FIG. 1. In the experiment leading to FIG. 1, the first PDH cycle was done after regeneration at 630° C., whereas later cycles were done after regeneration at 555° C. The temperature during the PDH was the same in all the cases, more specifically 555° C. A distinct decrease in activity upon recycling at a lower regeneration temperature can be seen for a Pt/Ga catalyst (Catalyst A in FIG. 1). The addition of 0.1% Ce (catalyst A-oxidation promoter in FIG. 1) results in a smaller decrease in activity upon lowering the regeneration temperature. This finding indicates that Ce is able to promote oxidation of the catalyst, and thereby it is possible to regain a larger part of the activity that was lost during the PDH.

### EXAMPLE 3

FIG. 2 shows the activity of catalysts with and without Ce. More specifically, FIG. 2 shows the results from testing 0.75 g of catalyst pellets in a single-pellet string reactor.

Catalyst B is the reference Pt/Ga catalyst on a carrier calcined at 1000° C. In the first experiment, the catalyst was regenerated every time at 630° C. for 2 hours. With this treatment, the catalyst reached its maximum potential. In the second experiment, the same catalyst was regenerated every time at 630° C. for 30 minutes. It can be seen that the activity is substantially lower in this case.

In the following experiments 3 to 6, Ce in an amount of 0.05, 0.1, 0.2 or 0.4 wt %, respectively, was co-impregnated with Pt/Ga. The testing was, in all cases, carried out with regeneration at 630° C. for 30 minutes. The performance of the catalyst with 0.05 wt % Ce is significantly better than that of Catalyst B under the same conditions. It actually comes close to the maximum potential activity of Catalyst B which is obtained after regeneration for 2 hours. It seems that although cerium improves the regeneration, it might also lower the maximum potential activity by blocking the active Ga sites. This suggests that ultimately, for the final catalyst, an optimal balance between maximum potential activity and regeneration speed has to be determined.

The two last experiments were done without any Pt in the catalyst. The second to last catalyst contains 0.1 wt % Ce, whereas the last catalyst contains no Ce. The absence of Pt resulted in a much lower activity, and the addition of Ce to the Ga catalyst without Pt did not improve the activity. The current view is therefore that Pt mainly promotes the dehydrogenation of propane, whereas Ce is promoting the regeneration of the catalyst without having any active role in the PDH step. The addition of cerium also does not have any effect on the selectivity or the oil or coke formation on the catalyst.

1. A catalyst for the dehydrogenation of lower alkanes, whereby the alkanes are dehydrogenated to the corresponding alkenes according to the reaction
CₙH₂ₙ₊₂<->CₙH₂ₙ+H₂
in which n is an integer from 2 to 5, by feeding the alkane to a catalyst-containing dehydrogenation reactor, wherein
the catalyst is based on optionally Si-doped alumina that has been impregnated with gallium and platinum, and
cerium in an amount from 0.001 to 0.5 wt % is added to the catalyst as an oxidation promotor together with gallium and platinum, thereby improving the regeneration of the catalyst composition.

2. (canceled)

3. The catalyst according to claim 1, wherein the amount of cerium added to the catalyst composition is between 0.05 and 0.2 wt %.

4. The catalyst according to claim 1, wherein cerium is added as a salt.

5. The catalyst according to claim 1, wherein cerium is added by impregnation together with gallium and platinum.

6. The catalyst according to claim 1, wherein the amount of platinum impregnated into the catalyst composition is up to around 200 ppm.

7. The catalyst according to claim 1, wherein cerium is added as Ce(NO₃)₂.6H₂O.
